(19) United States
(12) Patent Application Publication
(10) Pub. No.: US 2019/0321411 A1
(43) Pub. Date: Oct. 24, 2019

(54) METHOD FOR PROMOTING BIOACTIVE FACTORS IN PLACENTA TISSUE AND PRODUCT THEREOF

(71) Applicant: HCT Regenerative Co., Ltd., New Taipei (TW)

(72) Inventor: Yi-Yang James TSAI, New Taipei (TW)

(21) Appl. No.: 16/055,731

(22) Filed: Aug. 6, 2018

(30) Foreign Application Priority Data
Apr. 23, 2018 (TW) 107113701

(51) Int. Cl. A61K 35/50; A61K 38/39; A61L 26/00

(52) U.S. Cl. CPC A61K 35/50; A61K 38/39; A61L 27/24; A61L 26/0095; A61L 26/0033

(57) ABSTRACT

A method for processing placental tissue, including steps of: exposing a placenta tissue sample in a weak acid environment to obtain a first placenta tissue semi-finished product; performing a flushing procedure for the first placenta tissue semi-finished product to obtain a second placenta tissue semi-finished product, wherein the flushing procedure includes a first flushing procedure using a neutral buffer, a disinfection procedure using a mixture of the neutral buffer and at least one antibiotic, and a second flushing procedure using the neutral buffer to remove the at least one antibiotic; and making the second placenta tissue semi-finished product dehydrated or frozen in extremely low temperatures to obtain a final placenta tissue product.

### BACKGROUND OF THE INVENTION

### Field of the Invention

The present invention relates to a placenta tissue processing method, especially to a method for promoting bioactive factors in a placental tissue.

### Description of the Related Art

Human placenta tissue has been used in various reconstructive surgery procedures. In a wide range of clinical applications, such as ophthalmic procedures, wound healing, soft tissue repair, scar formation, and adhesion barriers, placental tissue has been used as a substrate material to be of great help. The placenta tissue is typically frozen or dehydrated before being stored for clinical use. After obtaining an agreement with a mother, a human placenta tissue is usually collected during a selective caesarean section surgery. The placenta tissue used for clinical use is composed of one or more of the following tissues, such as amniotic membrane, villus membrane, umbilical cord, amniotic fluid and amniotic stem cells.

The placenta tissue matrix is made up of collagen material layers of type I, III, IV, V, VI and VII, and cell adhesion growth factors of fibronectin and laminin.

As the collagen matrix of placenta tissue can provide biological active factor or growth factor, and contains protein molecules that stimulate cell arowth and differentiation, therefore, it can provide excellent transplant effect in surgical operations, and has seen more and more applications in regenerative medicine. Different types of growth factors can significantly accelerate the regeneration and repair of bone tissue, skin, cornea, cartilage, blood vessels or other tissues.

Generally, growth factors are derived from physiological solutions or cells containing extracellular matrix. Once obtained from the cells, only a small number of the growth factors are combined with the collagen matrix through electrostatic interactions, while most growth factors are washed away in the solution. Therefore, the growth factor concentration of general placenta tissue is very limited.

To solve the foregoing problems, a novel method for processing placental tissue is needed.

### SUMMARY OF THE INVENTION

One objective of the present invention is to provide a method for processing a placental tissue to make it contain a large number of bioactive factors by placing the placenta tissue in a weak acid environment to substantially promote the numbers of bioactive factors bound on a collagen matrix thereof.

Another objective of the present invention is to provide a method for processing a placental tissue to enhance the binding of bioactive factors on a collagen matrix thereof by adding heparin or heparin salt, so that the placenta tissue can contain a large number of bioactive factors.

Still another objective of the present invention is to provide a method for processing a placental tissue to enhance the binding of bioactive factors on a collagen matrix thereof by placing the placenta tissue in a weak acid environment containing heparin or heparin salts, so that the placenta tissue can contain a large number of bioactive factors.

To attain the foregoing objectives, a method for processing placental tissue is proposed, including steps of exposing a placenta tissue sample in a weak acid environment to obtain a first placenta tissue semi-finished product;

performing a flushing procedure for the first placenta tissue semi-finished product to obtain a second placenta tissue semi-finished product, wherein the flushing procedure includes a first flushing procedure using a neutral buffer, a disinfection procedure using a mixture of the neutral buffer and at least one antibiotic, and a second flushing procedure using the neutral buffer to remove the at least one antibiotic; and

making the second placenta tissue semi-finished product dehydrated or frozen in extremely low temperatures to obtain a final placenta tissue product.

In one embodiment, the placental tissue sample contains one or more components selected from a group consisting of amniotic, chorionic, umbilical, amniotic and amniotic stem cells.

In one embodiment, the weak acid environment has an acid-base value ranging between one and seven and contains one or more substances selected from a group consisting of acetic acid, glacial acetic acid, citrate and hydrochloric acid.

To attain the foregoing objectives, another method for processing placental tissue is proposed, including steps of:

exposing a placenta tissue sample in a heparin or heparin salt environment to obtain a first placenta tissue semi-finished product;

using a neutral buffer to flush the first placenta tissue semi-finished product to obtain a second placenta tissue semi-finished product; and

using a mixture of the neutral buffer and at least one antibiotic to perform a disinfection procedure on the second placenta tissue semi-finished product, and then using the neutral buffer to flush the second placenta tissue semi-finished product to remove the at least one antibiotic, and then dehydrating the second placenta tissue semi-finished product or placing the second placenta tissue semi-finished product in extremely low temperatures to obtain a final placenta tissue product.

In one embodiment, the placental tissue sample contains one or more components selected from a group consisting of amniotic, chorionic, amniotic and amniotic stem cells.

To attain the foregoing objectives, still another method for processing placental tissue is proposed, including steps of:

exposing a placenta tissue sample in a weak acid environment containing heparin or heparin salt to obtain a first placenta tissue semi-finished product;

using a neutral buffer to flush the first placenta tissue semi-finished product to obtain a second placenta tissue semi-finished product; and

using a mixture of the neutral buffer and at least one antibiotic to perform a disinfection procedure on the second placenta tissue semi-finished product, and then using the neutral buffer to flush the second placenta tissue semi-finished product to remove the at least one antibiotic, and then dehydrating the second placenta tissue semi-finished product or placing the second placenta tissue semi-finished product in extremely low temperatures to obtain a final placenta tissue product.

In one embodiment, the placental tissue sample contains one or more components selected from a group consisting of amniotic, chorionic, umbilical, amniotic and amniotic stem cells.

In one embodiment, the weak acid environment has an acid-base value ranging between one and seven and contains one or more substances selected from a group consisting of acetic acid, glacial acetic acid, citrate and hydrochloric acid.

In addition, the present invention also proposes a placenta tissue product, which contains the final placental tissue product produced by one of the methods for processing placental tissue described above.

To make it easier for our examiner to understand the objective of the invention, its structure, innovative features, and performance, we use preferred embodiments together with the accompanying drawings for the detailed description of the invention.

### BRIEF DESCRIPTION OF THE DRAWINGS

FIG. 1 illustrates a flowchart for an embodiment of the method of processing placenta tissue of the present invention.

FIG. 2 illustrates a flowchart for another embodiment of the method of processing placenta tissue of the present invention.

FIG. 3 illustrates a flowchart for still another embodiment of the method of processing placenta tissue of the present invention.

### DETAILED DESCRIPTION OF THE PREFERRED EMBODIMENTS

The principle of the invention is described below. The biological active factor, or named as growth factor, can come from a physiological solution or a cell containing an extracellular matrix. In a placenta tissue, the physiological solution is amniotic fluid, and the extracellular matrix consists of solid collagen, including amniotic membranes, chorionic villi, umbilical cord and amniotic stem cells.

The combination of two compounds of the growth factor and the collagen matrix can be further promoted by inducing electrostatic charges to the two compounds. Compounds have multiple charging nodes, and the addition of heparin can induce electrostatic charges to the compounds to further enhance the binding of the compounds. Besides, by adding a neutral buffer in the system, the combination of the compounds can be stabilized. Therefore, the present invention places the placenta tissue under an acidic condition (PH values are between 4-7 or 1-4) to promote the binding of the growth factors on the collagen matrix, or places the placenta tissue under a neutral condition and then adds heparin or heparin to promote the binding of the growth factors on the collagen matrix, or puts the placenta tissue under an acidic condition, and then adds heparin or heparin salts to further promote the combination of the growth factors and the collagen matrix.

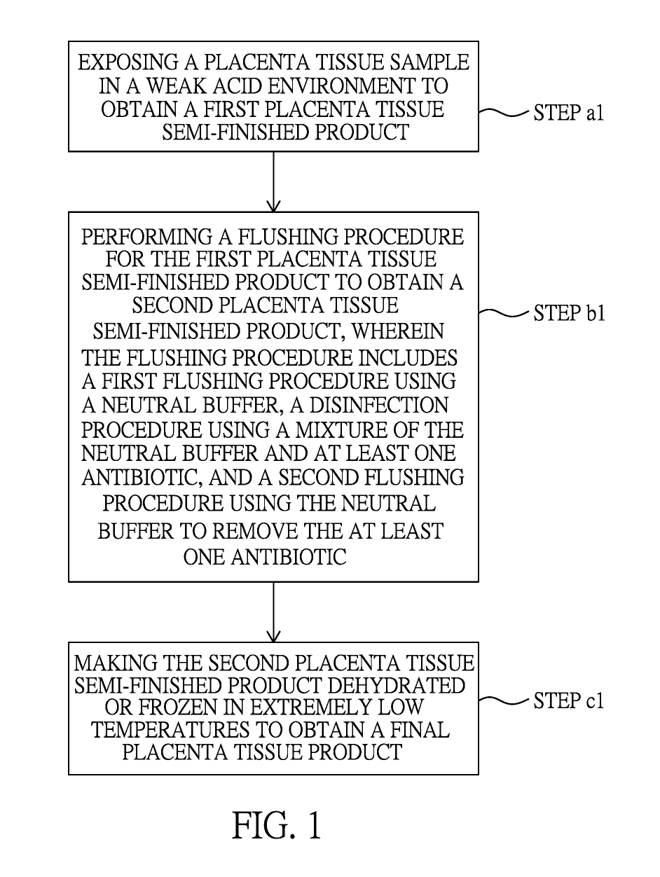

Refer to FIG. 1, which illustrates a flowchart of an embodiment of the method of processing placenta tissue of the present invention. As shown in the FIG. 1, the method of processing placenta tissue consists of the following steps: exposing a placenta tissue sample in a weak acid environment to obtain a first placenta tissue semi-finished product (step a1); performing a flushing procedure for the first placenta tissue semi-finished product to obtain a second placenta tissue semi-finished product, wherein the flushing procedure includes a first flushing procedure using a neutral buffer, a disinfection procedure using a mixture of the neutral buffer and at least one antibiotic, and a second flushing procedure using the neutral buffer to remove the at least one antibiotic (step b1); and making the second placenta tissue semi-finished product dehydrated or frozen in extremely low temperatures to obtain a final placenta tissue product (step c1).

In the step a1, the placental tissue sample contains one or more constituents selected from a group consisting of amniotic, chorionic, umbilical, amniotic and amniotic stem cells, and the acid-base value of the weak acid environment is between one and seven, and the weak acid environment can be made from acetic acid, glacial acetic acid, citrate, or hydrochloric acid.

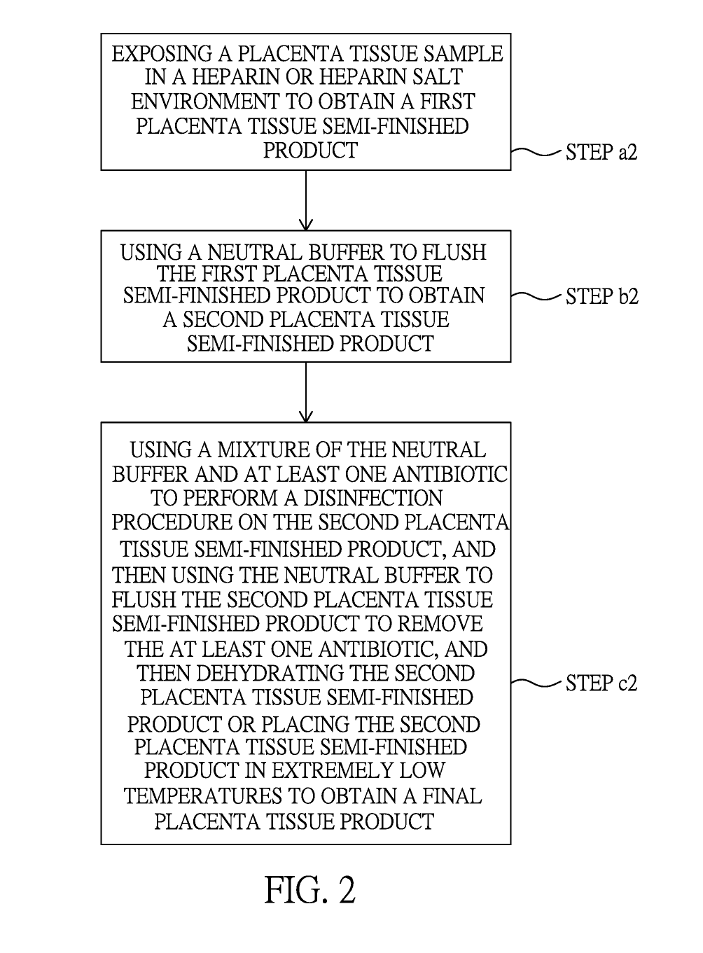

Refer to FIG. 2, which illustrates a flowchart of another embodiment of the method of processing placenta tissue of the present invention. As shown in the FIG. 2, the method of processing placenta tissue consists of the following steps: exposing a placenta tissue sample in a heparin or heparin salt environment to obtain a first placenta tissue semi-finished product (step a2); using a neutral buffer to flush the first placenta tissue semi-finished product to obtain a second placenta tissue semi-finished product (step b2); and using a mixture of the neutral buffer and at least one antibiotic to perform a disinfection procedure on the second placenta tissue semi-finished product, and then using the neutral buffer to flush the second placenta tissue semi-finished product to remove the at least one antibiotic, and then dehydrating the second placenta tissue semi-finished product or placing the second placenta tissue semi-finished product in extremely low temperatures to obtain a final placenta tissue product (step c2).

In the step a2, the placental tissue sample contains one or more constituents selected from a group consisting of amniotic, chorionic, umbilical, amniotic and amniotic stem cells.

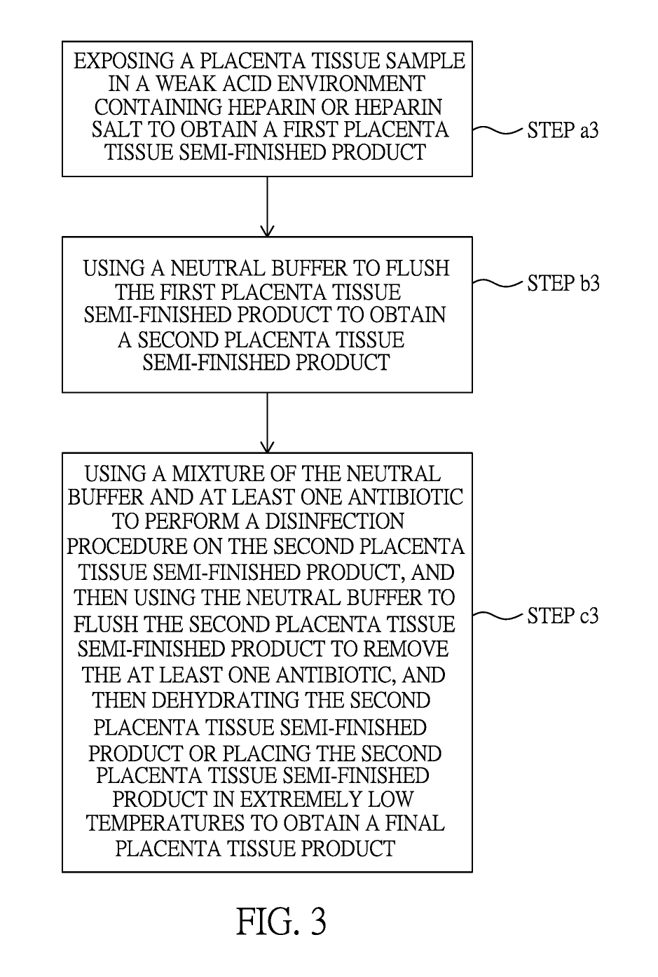

Refer to FIG. 3, which illustrates a flowchart of still another embodiment of the method of processing placenta tissue of the present invention. As shown in the FIG. 3, the method of processing placenta tissue consists of the following steps: exposing a placenta tissue sample in a weak acid environment containing heparin or heparin salt to obtain a first placenta tissue semi-finished product; (step a3); using a neutral buffer to flush the first placenta tissue semi-finished product to obtain a second placenta tissue semi-finished product (step b3); and using a mixture of the neutral buffer and at least one antibiotic to perform a disinfection procedure on the second placenta tissue semi-finished product, and then using the neutral buffer to flush the second placenta tissue semi-finished product to remove the at least one antibiotic, and then dehydrating the second placenta tissue semi-finished product or placing the second placenta tissue semi-finished product in extremely low temperatures to obtain a final placenta tissue product (step c3).

In the step a3, the placental tissue sample contains one or more constituents selected from a group consisting of amniotic, chorionic, umbilical, amniotic and amniotic stem cells, and the acid-base value of the weak acid environment is between one and seven, and the weak acid environment can be made from acetic acid, glacial acetic acid, citrate, or hydrochloric acid.

In accordance with the methods of processing placental tissue disclosed above, the present invention can thereby provide a placenta tissue product containing a large number of bioactive factors.

Thanks to the arrangements disclosed above, the present invention has the following advantages:

1. The method of processing placenta tissue of the present invention can make a placenta tissue contain a large number of bioactive factors by placing the placenta tissue in a weak acid environment to substantially promote the numbers of bioactive factors bound on a collagen matrix thereof.

2. The method of processing placenta tissue of the present invention can enhance the binding of bioactive factors on a collagen matrix of a placenta tissue by adding heparin or heparin salt, so that the placenta tissue can contain a large number of bioactive factors.

3. The method of processing placenta tissue of the present invention can enhance the binding of bioactive factors on a collagen matrix of a placenta tissue by placing the placenta tissue in a weak acid environment containing heparin or heparin salts, so that the placenta tissue can contain a large number of bioactive factors.

While the invention has been described by way of example and in terms of preferred embodiments, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

In summation of the above description, the present invention herein enhances the performance over the conventional structure and further complies with the patent application requirements and is submitted to the Patent and Trademark Office for review and granting of the commensurate patent rights.

What is claimed is:

1. A method for processing placental tissue, comprising steps of:
exposing a placenta tissue sample in a weak acid environment to obtain a first placenta tissue semi-finished product;
performing a flushing procedure for the first placenta tissue semi-finished product to obtain a second placenta tissue semi-finished product, wherein the flushing procedure includes a first flushing procedure using a neutral buffer, a disinfection procedure using a mixture of the neutral buffer and at least one antibiotic, and a second flushing procedure using the neutral buffer to remove the at least one antibiotic; and
making the second placenta tissue semi-finished product dehydrated or frozen in extremely low temperatures to obtain a final placenta tissue product.

2. The method for processing placental tissue as disclosed in claim 1, wherein the placental tissue sample contains one or more components selected from a group consisting of amniotic, chorionic, umbilical, amniotic and amniotic stem cells.

3. The method for processing placental tissue as disclosed in claim 1, wherein the weak acid environment has an acid-base value ranging between one and seven and contains one or more substances selected from a group consisting of acetic acid, glacial acetic acid, citrate and hydrochloric acid.

4. A method for processing placental tissue, comprising steps of
exposing a placenta tissue sample in a heparin or heparin salt environment to obtain a first placenta tissue semi-finished product;
using a neutral buffer to flush the first placenta tissue semi-finished product to obtain a second placenta tissue semi-finished product; and
using a mixture of the neutral buffer and at least one antibiotic to perform a disinfection procedure on the second placenta tissue semi-finished product, and then using the neutral buffer to flush the second placenta tissue semi-finished product to remove the at least one antibiotic, and then dehydrating the second placenta tissue semi-finished product or placing the second placenta tissue semi-finished product in extremely low temperatures to obtain a final placenta tissue product.

5. The method for processing placental tissue as disclosed in claim 4, wherein the placental tissue sample contains one or more components selected from a group consisting of amniotic, chorionic, umbilical, amniotic and amniotic stem cells.

6. A method for processing placental tissue, comprising steps of:
exposing a placenta tissue sample in a weak acid environment containing heparin or heparin salt to obtain a first placenta tissue semi-finished product;
using a neutral buffer to flush the first placenta tissue semi-finished product to obtain a second placenta tissue semi-finished product; and
using a mixture of the neutral buffer and at least one antibiotic to perform a disinfection procedure on the second placenta tissue semi-finished product, and then using the neutral buffer to flush the second placenta tissue semi-finished product to remove the at least one antibiotic, and then dehydrating the second placenta tissue semi-finished product or placing the second placenta tissue semi-finished product in extremely low temperatures to obtain a final placenta tissue product.

7. The method for processing placental tissue as disclosed in claim 6, wherein the placental tissue sample contains one or more components selected from a group consisting of amniotic, chorionic, umbilical, amniotic and amniotic stem cells.

8. The method for processing placental tissue as disclosed in claim 6, wherein the weak acid environment has an acid-base value ranging between one and seven and contains one or more substances selected from a group consisting of acetic acid, glacial acetic acid, citrate and hydrochloric acid.

9. A placenta tissue product, comprising the final placenta tissue product produced by the method for processing placental tissue as disclosed in claim 1.

10. A placenta tissue product, comprising the final placenta tissue product produced by the method for processing placental tissue as disclosed in claim 2.

11. A placenta tissue product, comprising the final placenta tissue product produced by the method for processing placental tissue as disclosed in claim 3.

12. A placenta tissue product, comprising the final placenta tissue product produced by the method for processing placental tissue as disclosed in claim 4.

13. A placenta tissue product, comprising the final placenta tissue product produced by the method for processing placental tissue as disclosed in claim 5.

14. A placenta tissue product, comprising the final placenta tissue product produced by the method for processing placental tissue as disclosed in claim 6.

15. A placenta tissue product, comprising the final placenta tissue product produced by the method for processing placental tissue as disclosed in claim 7.

16. A placenta tissue product, comprising the final placenta tissue product produced by the method for processing placental tissue as disclosed in claim 8.
